(19) United States
(12) Patent Application Publication
(10) Pub. No.: US 2013/0149724 A1
(43) Pub. Date: Jun. 13, 2013

(54) SYSTEMS, DEVICES AND METHODS FOR MICROFLUIDIC CULTURING, MANIPULATION AND ANALYSIS OF TISSUES AND CELLS

(71) Applicants: Ashok C. Chander, New York, NY (US); David Kong, Lexington, MA (US)

(72) Inventors: Ashok C. Chander, New York, NY (US); David Kong, Lexington, MA (US)

(73) Assignee: Ashok Chander, New York, NY (US)

(21) Appl. No.: 13/682,710

(22) Filed: Nov. 20, 2012

Related U.S. Application Data
(63) Continuation-in-part of application No. PCT/US2011/005544, filed on Oct. 7, 2011.
(60) Provisional application No. 61/561,907, filed on Nov. 20, 2011.
(60) Provisional application No. 61/677,157, filed on Jul. 30, 2012.
(60) Provisional application No. 61/391,340, filed on Oct. 8, 2010.

(51) Int. Cl. G01N 33/50

(52) U.S. Cl. CPC G01N 33/5008
USPC 435/7.92; 435/287.2

(57) ABSTRACT

Microfluidic devices for dissociating tissue, culturing, separating, manipulating, and assaying cells and methods for using the device are disclosed. Individual modules for tissue dissociation, cell, protein and particle separation, cell adhesion to functionalized, permissive micro- and nano-substrates, cell culturing, cell manipulation, cell and extracellular component assaying via metabolic and therapeutic compounds, compound titration, cell transfection, and micro-ELISA are described. Specialized micro- and nano-substrates and their methods of fabrication are also described. An integrated device is also disclosed. The devices and methods can be used for diagnostic applications, monitoring of disease progression, analysis of disease recurrence, compound discovery, compound validation, drug efficacy screening, and cell-based assays.

### CROSS-REFERENCE TO RELATED APPLICATIONS

This application claims the benefit of priority of U.S. Provisional Application No. 61/561,907, filed Nov. 20, 2011, entitled “Systems, Devices and Methods for Microfluidic Culturing, Manipulation and Analysis of Tissues and Cells,” and U.S. Provisional Application No. 61/677,157, filed Jul. 30, 2012, entitled “Systems, Devices and Methods for Microfluidic Culturing, Manipulation and Analysis of Tissues and Cells,” the teachings of which are incorporated herein in their entireties. This application also claims the benefit of priority as a continuation-in-part of International Application Number PCT/US2011/055444, filed Oct. 7, 2011 and designating the U.S., entitled “Systems, Methods, and Devices for Measuring Growth/Oncogenic & Migration/Metastatic Potential,” which claims the benefit of priority to U.S. Provisional Application No. 61/391,340, filed Oct. 8, 2010, the teachings of which are incorporated herein in their entireties.

### FIELD

Systems, methods, and devices related to the field of medical testing/diagnostics, cell-based assays, and compound discovery are provided herein. In various aspects, systems, devices, and methods are provided for the determination of the growth, and/or oncogenic potential, migration rate, and/or metastatic potential of mammalian cells or patient's cells (e.g., cells obtained from biopsy). In some aspects, microfluidic tissue disassociation, cell, protein, and particle separation, cell manipulation, and assay devices and methods for using the same are provided. Exemplary applications include but are not limited to diagnostic and cell based assays.

### BACKGROUND

Primary cell culture that allows the study of native tissue samples derived from an organism. Culturing cells derived from organisms, can be useful and necessary for applications such as medical diagnostics, cell-based assays, for compound discovery and characterization.

For example, cancer diagnosis and identification of compounds for treatment of cancer are of great interest due to the widespread occurrence of the diseases, high death rate, and recurrence after treatment. According to National Vital Statistics Reports, from 2002 to 2006 the rate of incidence (per 100,000 persons) of cancer in Caucasians was 470.6, in people of African decent 493.6, in Asians 311.1, and Hispanics 350.6, indicating that cancer is wide-spread among all races. Lung cancer, breast cancer and prostate cancer were the three leading causes of death in the US, claiming over 227,900 lives in 2007 according to the NCI.

Survival of a cancer patient depends heavily on detection. As such, developing technologies applicable for sensitive and specific methods to detect cancer is an inevitable task for cancer researchers. Existing cancer screening methods include: 1. the Papanicolau test for women to detect cervical cancer and mammography to detect breast cancer, 2. prostate-specific antigen (PSA) level detection in blood sample for men to detect prostate cancer, 3. occult blood detection for colon cancer, and 4 endoscopy, CT scans, X-ray, ultrasound imaging and MRI for various cancer detection. These traditional diagnostic methods however are not very powerful when it comes to cancer detection at very early stages and give little prognostic information. Moreover, some of the screening methods are quite costly and not available for many people.

Likewise, existing methods for cancer staging are often qualitative and therefore limited in applicability. For example, diagnoses made by different physicians or of different patients using existing methods can be difficult to compare in a meaningful manner due to the subjective nature of these methods. As a result, the subjectivity of the existing methods of cancer staging often results in overly aggressive treatment strategies. By way of example, in the absence of better data, the most drastic, potentially invasive, strategy is often recommended, which can lead to overtreatment, poor patient quality of life, and increased medical costs.

One method to detect and/or characterize cancer, for example, is to directly assess living tissue derived from small biopsy samples taken from suspicious tissue. To get a relevant and useful sense of the biological characteristics of tissue, one would be well served by being able to culture biopsy tissue in vitro.

Therefore, the development of technology that is specific and reliable for culturing primary human tissue and/or detecting and characterizing a cancer (e.g., determining the growth, oncogenic, migration rate, and/or metastatic potential of cells obtained from a patient) is an area of significant importance. Likewise, there remains a need for improved systems, methods, and devices for diagnostic cell-based assays and compound discovery.

### SUMMARY

The systems, methods, and devices described herein generally involve medical testing/diagnostics, cell-based assays, and/or compound discovery. In various aspects, microfluidic devices, systems, and methods disclosed herein can provide clinical and/or research purposed diagnostics and assay platforms that enable tissue disassociation, cell, protein, and particle separation, and cell manipulation. The systems and devices disclosed herein can provide, for example, the culturing of a small number of cells in environments that can approximate in vivo conditions, while allowing for a determination of the cells' growth, and/or oncogenic potential, migration rate, and/or metastatic potential. A determination of these characteristics can, among other things, facilitate treatment decision steps taken by a physician for patients having symptoms of cancer and/or aid in the discovery of therapeutics that alter and/or perturb a cell's characteristics that engender its cancer-like, oncogenic, and/or metastatic phenotype.

For example, quantitative prognostic metrics according to aspects of the invention can improve the accuracy of diagnosis by supplementing a physician's decision-making process with clinical data to support the available treatment options. As a result, embodiments of the invention can provide numerous advantages, for example, reduced healthcare costs, reduced risk associated with treatment, improved patient quality of life, and increased patient survival.

As will be described in detail below, one exemplary aspect of the invention provides cell processing systems and devices that include microfluidic channels and a substrate to process (e.g., culture, filter, image) cells derived, for example, from a biopsy. In other aspects, the systems and devices enable diagnostic imaging, cell-based assays such as metabolic testing, and/or compound discovery.

In one exemplary embodiment, a system for cell processing is provided. The system can include at least one microfluidic cell dissociation module and at least one microfluidic cell-processing module fluidly coupled to the at least one cell dissociation module. The cell dissociation module can be configured, for example, to dissociate one or more tissue fragments received therein into one or more of single cells and/or smaller tissue fragments. The microfluidic cell-processing module can receive at least a portion of said one or more single cells and/or smaller tissue fragments. The system can additionally include at least one reservoir in communication with at least one of the dissociation module and the cell-processing module. The reservoir can be configured to store one or more reagents to be used within the dissociation module and/or the cell-processing module.

In various embodiments, one or more cell-processing modules of the system can be configured to perform various cell processing functions. In various aspects, microfluidic systems can incorporate one or more of the following exemplary individual microfluidic modules and/or substrates:
a cell dissociation module, which can receive mammalian tissue and separate the tissue into smaller clumps and/or single cells, e.g., via enzymatic, mechanical, and/or shear forces;
perfusion chambers, in which single cells and/or clumps of cells can be adhered to specialized micro- and nano-featured substrates. When functionalized with protein coatings, these specialized substrates can create a permissive surface for cell adhesion and subsequent examination via microscopy techniques. Cells can also be cultured in such an environment;
methods for fabricating these micro- and nano-featured substrates within microfluidic chambers;
perfusion layers, singular or arrayed, integrated above reaction chambers for the introduction of biomolecules and other compounds into the reaction chambers below. Other local environmental conditions such as temperature and gas partial pressure can also be controlled;
metabolic assay, compound discovery, and titration modules, whereby cells adhered to various substrates can be subjected to various compounds for assay or therapeutic applications. The cells can then be monitored via microscopy techniques for their response. Titrations can also be conducted in the titration module and can be similarly inspected via microscopy;
a cell separation module, where cells and extra-cellular components such as proteins and other particles can be segregated and sorted;
a DNA transfection module where biomolecules can be inserted into cells for further assaying;
a micro-ELISA module where extra-cellular components can be assayed; and
various specialized substrates for cell adhesion and also for testing cellular properties such as invasion potential.

In an exemplary embodiment, a cell dissociation module can include a first cell dissociation chamber having at least one inlet port for receiving one or more tissue fragments and a channel fluidly coupled to said chamber to allow fluid to be circulated through the chamber. A pump can be coupled to any of the channel and the chamber to cause circulation of the fluid through the channel and/or chamber.

The cell dissociation module can have various configurations and dimensions. By way of example, the inlet port can have a maximum dimension of about 10 mm for receiving tissue fragments and/or the channel of the cell dissociation module can have a cross-sectional dimension in a range of about 10 microns to about 1000 microns. In some aspects, a plurality of microstructures disposed in the channel can facilitate dissociation of said one or more tissue fragments. The microstructures can have a variety of dimensions. For example, the microstructures can be a material structure having a size in at least one dimension, and in some cases in two or three dimensions, less than about 1000 microns. The microstructures can have a variety of configurations to facilitate dissociation, e.g., through mechanical perturbation. For example, the microstructures can be pyramidal.

In some aspects, the channel can include at least one inlet port for introducing one or more reagents therein. For example, the inlet port can be used to introduce reagents configured to facilitate dissociation of said one or more tissue fragments. The reagent(s) can be a protease, for example, such as trypsin, DNase, papain, collagenase type I, II, III, IV, hyoluronidase, elastase, protease type XIV, pronase, dispase I, dispase II, and neutral protease.

In some embodiments, the cell dissociation module can include a second dissociation chamber fluidly coupled to the first dissociation chamber via the channel so as to provide a closed loop fluid circulating path. In some aspects, the second dissociation chamber can include an outlet port, for example, that allows fluids, dissociated cells, and other particles to be transmitted to one or more downstream modules for further processing.

As noted above, systems and devices in accord with the present teachings can include a cell-processing module. In one exemplary embodiment, the cell-processing module can include an optically transparent layer having at least one portion transmissive to optical radiation and a cell-processing layer defining a microfluidic channel for receiving a fluid having cells suspended therein. The cell-processing layer can include one or more cell adhesion surfaces, upon which cells can preferentially adhere relative to surrounding areas of the cell processing layer. By way of example, the one or more cell adhesion surfaces can be functionalized with one or more reagents suitable for facilitating preferential adhesion of cells to said surfaces (e.g., fibronectin, collagen, laminin, and vitronectin). The optically transmissive portion can be positioned relative to one or more of the cell adhesion surfaces to allow optical interrogation of cells adhered to the cell adhesion surfaces. In some aspects, the microfluidic channel of the cell-processing module can be coupled directly or indirectly to a channel of the cell dissociation module.

In some embodiments, the one or more cell-processing modules further comprise at least one inlet port for introducing one or more reagents into said microfluidic channel. The reagents can be selected, for example, to facilitate at least one of metabolic assays and compound discovery.

In various aspects, the cell adhesion surfaces can have a variety of configurations. For example, one or more cell adhesion surfaces can be substantially planar. Alternatively, in some aspects, one or more cell adhesion surfaces can include one or more microstructures. For example, the microstructures can comprise microgaps extending into a substantially planar surface and/or pillars and columns.

In some aspects, the cell-processing module can additionally comprise a perfusion layer coupled to the cell processing layer, the perfusion layer comprising one or more channels disposed therein and positioned relative to the one or more cell adhesion surfaces so as to allow diffusion of any of a gas and a nutrient to cells disposed on the one or more cell adhesion surfaces. In some embodiments, the perfusion layer can include, for example, an inlet port and outlet port for ingress and egress of fluids and/or gases.

The cell-processing module can be made from a variety of materials. By way of example, the cell-processing layer of the cell-processing module can be any of thermoplastics, thermosets, and elastomers such as epoxy, phenolic, PDMS, glass, silicones, nylon, polyethylene, polysterene. The optically transparent layer can also be made of the same or different materials relative to that of the cell-processing layer. By way of example, the optically transparent layer can comprise glass.

In some aspects, at least one surface of the optically transparent portion can be functionalized with one or more reagents suitable to prevent adhesion of cells to said surface.

The cell-processing module can have a variety of dimensions. For example, the cell-processing layer can have a thickness in the range from about 1 microns to about 100,000 microns. In some aspects, the microfluidic channel of the cell-processing layer can comprise at least two opposed surfaces separated from one another by a distance in a range of about 0.001 micron to about 100,000 microns. In various embodiments, one of these opposed surfaces can be a surface of the optically transparent layer.

In some aspects, the systems for cell processing can include additional cell-processing modules. By way of example, the systems can include a sorter module fluidly coupled to the cell dissociation module and cell-processing module and disposed therebetween. The sorter module can be configured, for example, to discriminate particles based on size. In some embodiments, for example, the cell sorter module can be configured to divert cells having a diameter greater than about 10 microns to a downstream cell-processing module.

In some aspects, the system can include a module to enable enzyme-linked immunosorbent assays (ELISA). For example, an ELISA module can be fluidly coupled to the cell sorter module such that particles having a diameter less than about 10 microns are diverted to said ELISA module. In some aspects, the ELISA module can comprise one or more surfaces functionalized with high affinity biomolecules.

In some embodiments, the system can include a titration module.

In various embodiments, the system can include at least a first and a second cell-processing modules connected in series, wherein each of the first and second cell-processing modules comprises at least one cell adhesion surface, and wherein a cell adhesion surface of the first cell-processing module differs from at least one of the cell adhesion surfaces of the second cell-processing module. In some embodiments, the one or more cell-processing modules can additionally or alternatively include at least first and second cell-processing modules connected in parallel.

In some aspects, the cell processing system can also include an imager configured to interrogate cells within the cell-processing module(s). For example, the imager can be configured to image a cell adhesion surface of the cell-processing module with one of fluorescence, confocal, differential interference contrast, and total internal reflection fluorescence microscopy.

Methods for processing tissue and/or cells are also provided. In one exemplary embodiment, a method of processing tissue is provided that includes introducing one or more tissue fragments into a microfluidic cell dissociation module such that said one or more tissue fragments dissociate into any of single cells and smaller tissue fragments. At least a portion of said single cells and/or smaller tissue fragments can be transferred to a microfluidic cell-processing module fluidly coupled to said at least one cell dissociation module. The cells and/or smaller tissue fragments can be processed such that the cells adhere to one or more cell adhesion surfaces of the microfluidic cell-processing module. In some aspects, at least a portion of said cells adhered to one of said cell adhesion surfaces can be imaged.

Additional modules can also be provided to perform various other cell processing steps. By way of example, the methods can additionally enable one or more of the following: separating particles between about 1 and about 50 microns in diameter; sustaining growth and division of said cells; measuring enzymatic activity of said cells; measuring protein content of tissue mass; measuring one or more biomarkers from cells; culture parts of said tissue fragments and cells derived from said tissue fragments; lysing said cells; analyzing cell lysates; and enriching single or multiple cell types.

In some aspects, the various modules described herein (or at least a portion of the modules such as the microfluidic channels) can be formed in a monolithic substrate. For example, a cell-dissociation module and the cell-processing layer of a cell-processing module can be formed in a monolithic substrate. Such devices can be fabricated and operated with techniques familiar to those skilled in the art of multi-layer soft lithography, photolithography, and microfluidic device fabrication and in light of the teachings herein.

In addition or in the alternative, because discrete functions can be performed by the one or more modules, individual modules can be coupled to one another and/or combined to create an integrated chip or platform that can be used for numerous biological and chemical applications, for example, but not limited to a cell-based assay for compound discovery, validation, testing, and or an in vitro diagnostic or prognostic test for disease states such as epithelial-born cancers, blood-born cancers, bone cancer, skin cancer, lung cancer, prostate cancer, breast cancer, pancreatic cancer, brain cancer, cervical cancer, colon cancer, stomach cancer, cardiac hypertrophy, cardiovascular diseases, and fibrotic diseases such as fibrosis of the kidney, and liver.

By disassociating and or culturing tissue and cells derived from an organism using any combination of the devices and substrates described herein, it can be possible to create powerful experimental and diagnostic tools with immediate research, pharmaceutical, biotechnology, and clinical development applications.

These and other embodiments, features, and advantages will become apparent to those skilled in the art when taken with reference to the following more detailed description of various exemplary embodiments of the invention in conjunction with the accompanying drawings.

### BRIEF DESCRIPTION OF THE DRAWINGS

FIG. 1 is a schematic representation of a microfluidic tissue dissociation chamber.

FIG. 2 is a schematic representation of a microfluidic tissue dissociation chamber.

FIG. 3 is a schematic representation of a microfluidic tissue dissociation chamber.

FIG. 4 is a schematic representation of a microfluidic tissue dissociation chamber.

FIG. 5 is a schematic representation of a microfluidic tissue dissociation chamber.

FIG. 6 is a schematic representation of a microfluidic perfusion chamber designed for the introduction and adhesion of cells for inspection via techniques such as optical-based microscopy.

FIG. 7 is a schematic representation of a mold from which a microfluidic perfusion chamber can be cast.

FIG. 8 is a schematic representation of a microfluidic perfusion chamber featuring a perfusion layer.

FIG. 9 is a schematic representation of a microfluidic perfusion chamber featuring multiple perfusion channels.

FIG. 10A is a schematic representation of a microfluidic device enabling the adhesion and release of cells from an array of perfusion chambers.

FIG. 10B is a schematic representation of a microfluidic device featuring an array of perfusion chambers for high throughput analysis.

FIG. 11 is a schematic representation of a microfluidic device enabling the inspection of cells upon being subjected to various metabolic assays and therapeutic compounds in conjunction with a titration system.

FIG. 12 is a schematic representation of a microfluidic DNA transfection module where biomolecules can be introduced into adhered cells.

FIG. 13 is a schematic representation of a microfluidic cell, protein, and particle segregation and separation module.

FIG. 14 is a schematic representation of a microfluidic micro-ELISA module.

FIG. 15 is a schematic representation of a micro-nano substrate.

FIG. 16 is a schematic representation of a micro-nano substrate.

FIG. 17 is a schematic representation of a micro-nano substrate.

FIG. 18 is a schematic representation of a micro-nano substrate.

FIG. 19 is a schematic representation of a micro-nano substrate.

FIG. 20 is a schematic representation of a micro-nano substrate.

FIG. 21 is a schematic representation of a micro-nano substrate.

FIG. 22 is a schematic representation of a micro-nano substrate.

FIG. 23 is a schematic representation of a micro-nano substrate.

FIG. 24 is a schematic representation of an integrated microfluidic device featuring a tissue dissociation module, a cell, protein, and particle segregator/sorter module, a micro-ELISA module, a perfusion chamber array featuring various micro- and nano-substrates, a metabolic assay and compound discovery module, and a titration module.

FIG. 25 is a schematic representation of an integrated microfluidic device featuring inlets, sample dissociation module, sample sorting and enriching module, and perfusion array.

FIG. 26 depicts an exemplary embodiment of an integrated microfluidic system in accord with various aspects of the present teachings.

### DETAILED DESCRIPTION

The following detailed description should be read with reference to the drawings. The drawings, which are not necessarily to scale, depict selected embodiments and are not intended to limit the scope of the invention. The detailed description illustrates by way of example, and is not intended to limit the scope of the invention.

Unless defined otherwise, technical and scientific terms used herein have the same meaning as commonly understood by one of ordinary skill in the art to which the invention belongs. Singelton et al., Dictionary of Microbiology and Molecular Biology 2^{nd} ed., J. Wiley & Sons (New York, N.Y. 1994); and Webster's New World Medical Dictionary, 2^{nd} Edition, Wiley Publishing Inc., 2003. provide one skilled in the art with a general guide to many of the terms used in the present application. Reference is also made to PCT/US2011/055444 filed Oct. 7, 2011, the contents of which are incorporated herein by reference in their entirety. For purposes of the present invention, the following terms are defined below.

The term “PDMS” refers to the silicone elastomer poly-dimethyl-siloxane.

The term “PMMA” refers to poly-methyl-methacrylate

The term “PEG” refers to poly-ethylene-glycol

The teachings herein generally provide microfluidic systems, devices, and methods whereby mammalian tissue and cells can be dissociated, cultured, manipulated, assayed, and/or inspected. As will be appreciated by a person skilled in the art, the systems, methods, and devices described herein can have application in medical testing/diagnostics, cell-based assays, and/or compound discovery. In various aspects, the exemplary microfluidic devices, systems, and methods can provide clinical and research purposed diagnostics and assay platforms that enable tissue disassociation, cell, protein, and particle separation, and other cell manipulation. By way of example, the present teachings can enable the culturing of a small number of cells in environments that can approximate in vivo conditions, while allowing for a determination of the cells' growth, and/or oncogenic potential, migration rate, and/or metastatic potential.

As will be described in detail below, exemplary cell processing systems and methods in accordance with the present teachings enable a variety of cell processing procedures, cell-based assays, and/or experiments (e.g., compound discovery) to be performed within the various microfluidic modules described in detail below. Though particular cell-processing functions are generally described with reference to individual cell-processing modules, it will be appreciated that the various exemplary modules and/or their functions can be combined to form a cell-processing system for performing multiple cell-processing functions. By way of example, it will be appreciated that various exemplary modules described herein can be combined in a single device (e.g., in a lock-and-key manner or combined in a monolithic microfluidic chip) to enable a specific clinical, diagnostic, and/or experimental workflow. Accordingly, the following description provides exemplary modules that can be incorporated into various systems in accord with the present invention.

### Tissue Dissociation

In one aspect, a microfluidic tissue dissociation module can be provided. Tissue disassociation involves the progressive isolation of smaller and smaller clusters of tissue and cell clumps into a single cell suspension. The process of disassociation can be accomplished via a number of methods and combinations of methods including, but not limited to, enzymatic treatment, mechanical agitation, stress and shear forces.

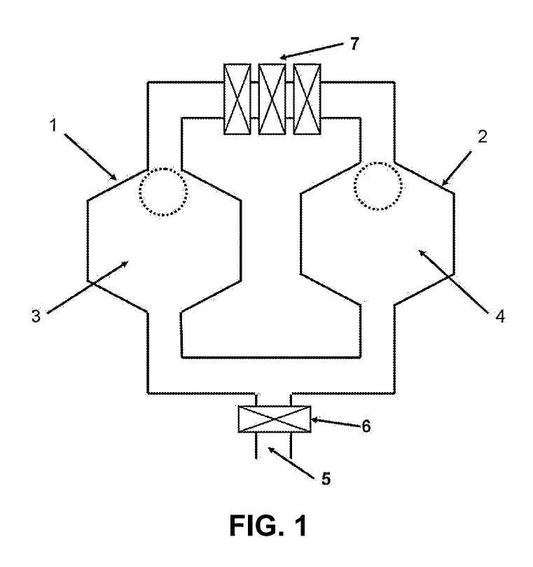

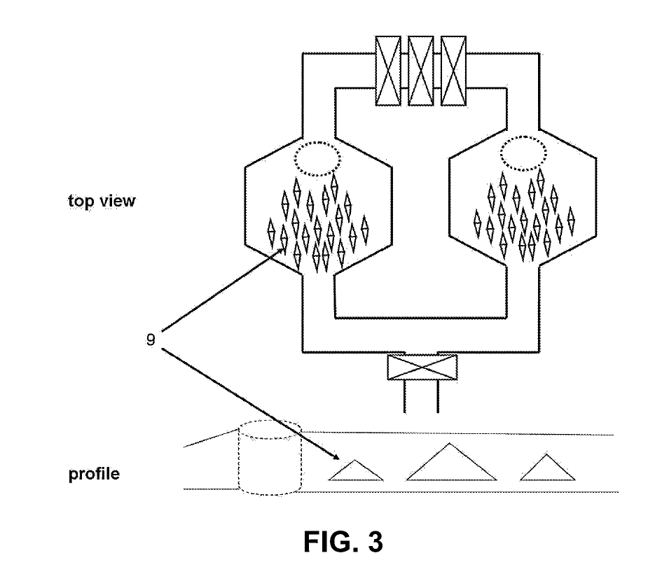

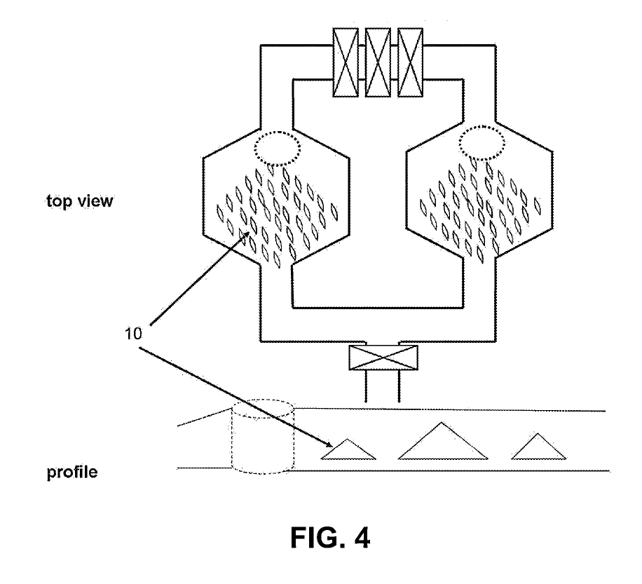

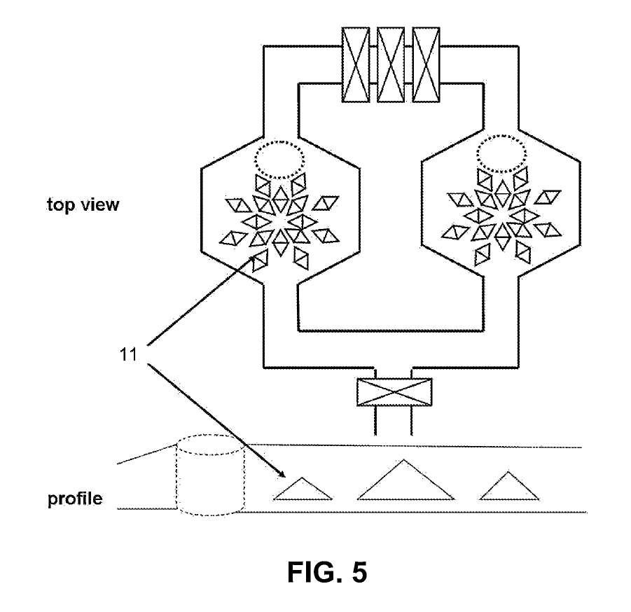

Schematic representations of microfluidic devices for tissue dissociation are shown in FIGS. 1-5. In an exemplary depiction, as seen in FIG. 1, two dissociation chambers 1 and 2 are utilized to reduce two input tissue samples to progressively smaller clumps of tissue until ultimately an approximately single cell suspension is achieved. Tissue fragments (e.g., minced tissue, tissue slices, etc.) can be introduced into the device via one or more inlet ports 3 and 4. As will be appreciated by a person skilled in the art, the tissue fragments can range in sizes, for example, in a range of about 0.001 mm to 10 mm. The inlet can be configured to accommodate a variety of tissue sizes. By way of example, the inlet port can be configured to have a maximum dimension of about 10 mm so as to limit the size of tissue fragment that can be introduced to the tissue dissociation module. In some embodiments, upon introduction of tissue into the inlets, the port(s) 3, 4 can be sealed with a small piece of PDMS, glass, epoxy, or other sealant. In some aspects, tissue samples can be injected into the device via needles.

Once loaded within the device, a dissociation formulation containing enzymes, detergents, proteins, salts, and other bio-compatible effectors can be introduced into the device via one or more channel(s) 5, and upon the closure of valve 6, mixing valves 7, when operated in a peristaltic fashion, can be utilized to circulate the dissociation formulation at varying flow rates. As will be appreciated by a person skilled in the art, any pumps known or developed hereafter and modified in accord with the teachings herein can be utilized to circulate the dissociation formulation within the dissociation module. Moreover, the various reagents introduced to aid tissue dissociation can be stored in a reservoir fluidly coupled to the channel 5 and/or dissociation chambers 1, 2. Example reagents include proteases such as protease selected from the group consisting of trypsin, DNase, papain, collagenase type I, II, III, IV, hyoluronidase, elastase, protease type XIV, pronase, dispase I, dispase II, neutral protease

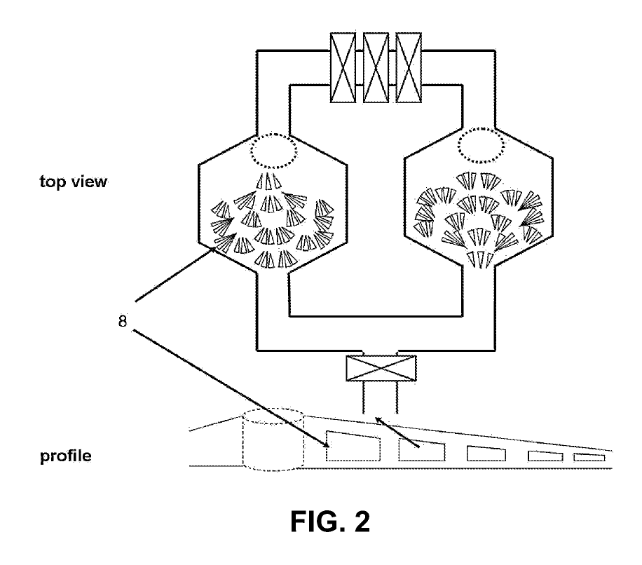

With reference now to FIGS. 2-5, various microstructures can be incorporated into the dissociation module to facilitate mechanical perturbation and/or mixing of the tissue fragments and dissociation reagents. In the depicted embodiments, for example, a plurality of microstructures are present on the “ceiling” or “floor” of the dissociation chamber to aid in the mechanical perturbation of the tissue samples. As will be appreciated by a person skilled in the art, microstructures 8, 9, 10, and 11 can have various dimensions, configurations, and geometries. By way of example, the microstructures 8 can range from about 1 micron to about 100 microns, and their presence, along with varying flow rates, and finally the presence of dissociation enzymes in the formulation, can enable the reduction of tissue fragments to single cells.

Upon completion of tissue dissociation into single cells or small clumps of cells, the dissociation module can, for example, transfer the cells and the associated fluid and other particles to one or more downstream modules for further processing such as manipulation, adhesion, culturing, assay, and inspection of these cells via the various preferred embodiments described below.

### Single Perfusion Chamber

As noted above, various cell-processing modules can be utilized to perform various functions. In an exemplary embodiments, a perfusion chamber module can be provided. Specialized substrates with micro- and nano-features coated with proteins solutions such as fibronectin, laminin, vitronectin and collagen can be incorporated within the perfusion chamber module so as to create permissive surfaces upon which mammalian cells can preferentially adhere and be cultured when they otherwise would be unable. Examples of these substrates are described, for example in PCT/US2011/055444 filed Oct. 7, 2011, the contents of which are incorporated herein by reference.

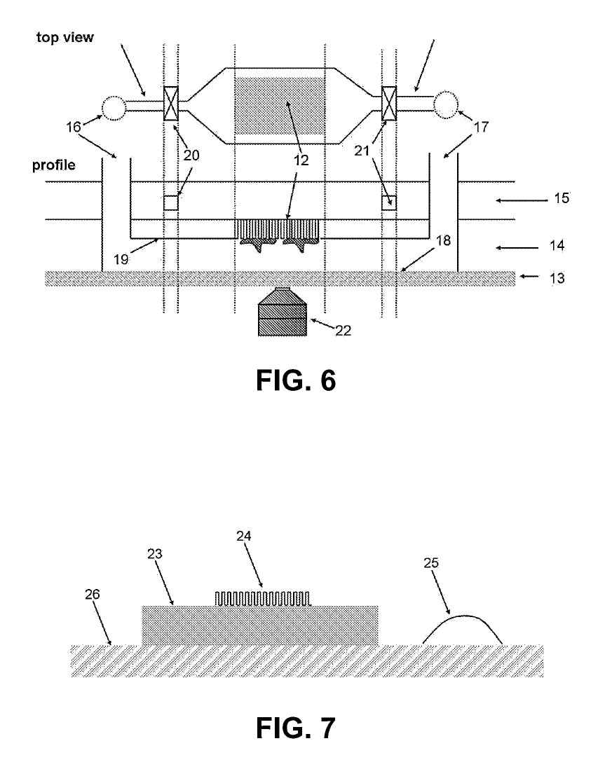

With reference now to the exemplary module depicted in FIG. 6, the single perfusion chamber can enable the introduction of mammalian cells, their adhesion to a specialized substrate region 12, and subsequent inspection via techniques such as optical-based microscopy. As noted above, this substrate region can be composed of micro- and nano-structures with features sized as small as nanometers and as large as microns to enable investigation of the cells' characteristics (e.g., motility), as will be discussed in detail below.

As will be appreciated by a person skilled in the art, the modules can have a variety of shapes and sizes and can comprise a variety of materials. By way of example, the depicted module comprises three layers: a featureless bottom layer 13, a middle layer 14 with channels through which cells and other biomolecules can be introduced and manipulated, and a top layer 15 with additional channels. The channels can have a variety of dimensions. By way of example, the channels can be microfluidic channels. In some aspects, the channels can have at least one dimension, and in some cases two or three dimensions, in the range from about 10 microns to about 1000 microns. In a preferred embodiment the bottom layer 13 can be composed of an optically clear material such as glass (e.g., with a thickness of about 100 microns), the middle layer 14 can be composed of PDMS (e.g., with a thickness of in the range from about 1 microns to about 100,000 microns, about 125 microns), and the top layer 15 can also be composed of PDMS (e.g., with a thickness of about 1 cm). In such an embodiment, the dissociated cells, cell culture media, and other biomolecules can be introduced into the device via inlet 16 and exit the device via outlet 17, with flow occurring in middle layer 14. It will be appreciated that the layers can also be composed of any thermoplastics, thermosets, elastomers such as epoxy, phenolic, PDMS, glass, silicones, nylon, polyethylene, any polysterene, or any other suitable material.

In this embodiment, the device can be operated in the following manner. First, the middle layer of the multi-layer device having the microchannels formed therein can be produced utilizing techniques well known in the art (e.g., soft lithography, injection molding) and modified in accord with the teaching herein. For example, the middle layer can comprise PDMS and the lower layer can comprise glass, silane chemistry can be utilized to functionalize the glass “floor” of the device 18 along with a passivating molecule such as PEG to create an un-permissive surface for cell adhesion. Next, an aqueous solution containing proteins such as fibronectin, collagen, laminin, and vitronectin, can be introduced and incubated to functionalize the PDMS “ceiling” 19 of the perfusion chamber, including importantly the substrate region 12. This incubation can be conducted at 37 degrees Celsius, between 1 and 48 hours, at concentrations that saturate the surface area of the substrate (˜10 ug/ml) thus resulting in a surface amenable to mammalian cell adhesion. Combinations of these proteins can also be utilized.

After functionalizing both the “floor” 18 (glass with PEG via silane chemistry) and “ceiling” 19 (PDMS with protein formulation), the dissociated mammalian cells in bio-compatible cell culture media can be introduced into the chamber via inlet 16 with valve 20. Any air in the perfusion chamber can be removed by closing valve 21 and applying pressure to the solution via inlet 16, resulting in the diffusion of any air bubbles from the bulk PDMS. Next, cells in solution will preferentially adhere to the specialized substrate 12. Once adhered, the cells can be imaged via objective 22, following the addition of reagents, for example, that enable the quantification of biomarkers. Examples of these biomarkers are described, for example in PCT/US2011/055444 filed Oct. 7, 2011, the contents of which are incorporated herein by reference.

In the case of use as a diagnostic device, for example, cancer cells, which could be dissociated from biopsy tissue and can be, for example, prostate cancer, can adhere to the substrate region for imaging via techniques such as fluorescence, confocal, differential interference contrast (DIC), and total internal reflection fluorescence (TIRF) microscopy utilizing objective 22. It should be appreciated that any imaging technique known in the art or hereafter developed and modified in accord with the present teachings can be used to interrogate the cells. Biomarker data acquired from such imaging, as described, for example, in PCT/US2011/055444, can be utilized to provide quantitative metrics that assess and predict the aggressiveness, or oncogenic potential, of the cancer, as well as the motility, or metastatic potential, of the cancer. The types of cells that can be studied in such a device include, but are not limited to, breast, epithelial, lung, skin, and pancreatic cancers as well as cardiac hypertrophy.

Cells adhered to the cell adhesion surface (i.e., substrate 12) can be further cultured for extended periods of times by conducting washes and exchanges in the perfusion chamber by, for example, removing the culture media via outlet 17 and replacing it with fresh biocompatible cell culture media. Such exchanges can be conducted, for example, every 12 hours. The “used” cell culture media can contain growth factors and other compounds excreted by the cells that promote cell growth; thus, some of this “used” media can be re-circulated back into the chip in conjunction with freshly prepared media.

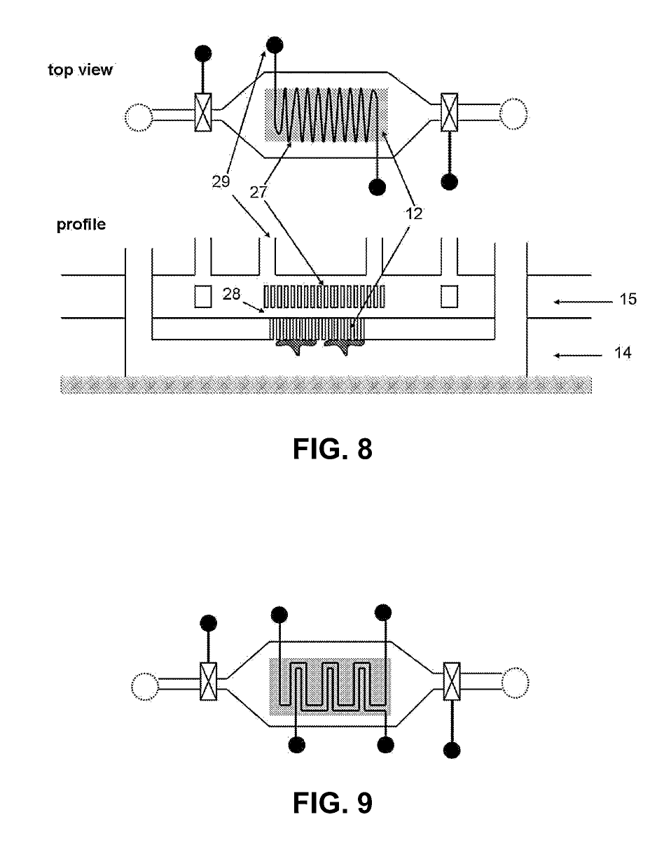

Additionally, the entire device can be kept in an environmental chamber to control conditions such as temperature and gas partial pressures to best facilitate cell culture and growth. For example, the chip can be maintained at 37 C and 5% CO2. Local environmental control can be maintained via the perfusion layer shown in FIG. 8 and discussed below.

Furthermore, other fluids can be introduced into the perfusion chamber(s) for interaction with adhered cells, including but not limited to nanotubes, nanorods, quantum dots, and fluids such as wash buffers, cellular fixing solutions and detergents such as triton.

While in the previous description the middle and top layers (14, 15) are composed of PDMS, the middle and top layers can also be composed of deformable or soft materials such as plastics or polymers such as PMMA, or hard materials such as glass or silicon.

### Specialized Substrate Fabrication

As noted above, the modules and/or their microchannels can be produced using a variety of techniques such as lithography or injection molding. In one aspect, for example, the specialized substrate 12 shown in FIG. 6 can be fabricated in a number of ways. In a preferred embodiment, the substrate can be cast from a mold as shown in FIG. 7. Here, multiple layers of photoresist 23, 24, and 25 are utilized to create features of various geometries and heights on substrate 26.

Specifically, the perfusion chamber itself could be cast from a silicon mold featuring a negative photoresist such as SU-8 to define the dimensions of the chamber 23, while the micro- and nano-structures 24 can be defined by a positive photoresist such as an AZ electronic materials series resist. A negative resist could also be utilized. Channels 25 for flowing cells and other biomolecules could be cast from positive photoresists suitable for reflow such as AZ 50XT.

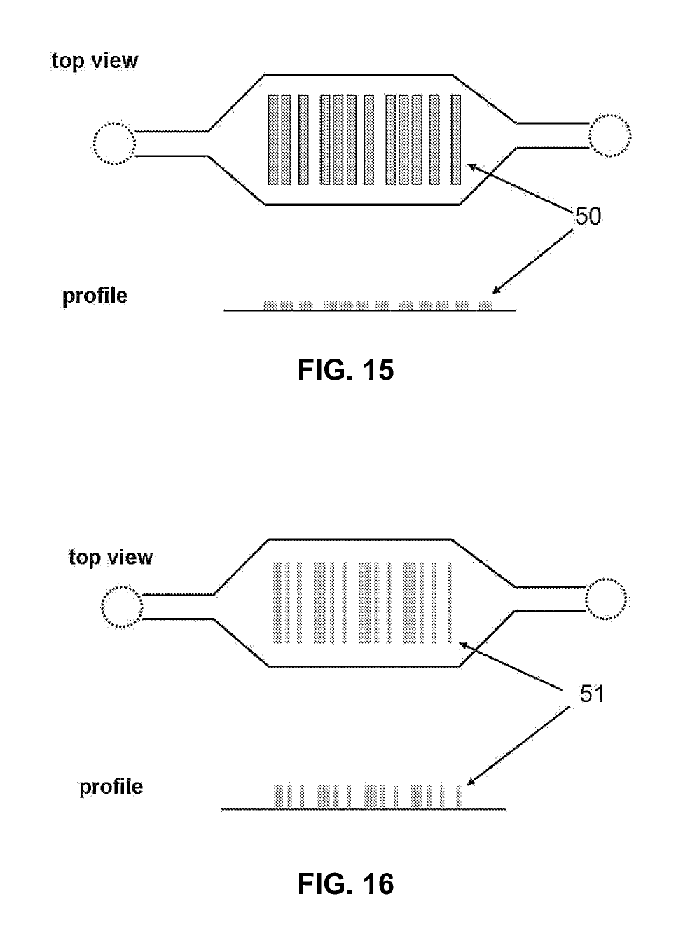

In a preferred embodiment, the micro- and nano-structures 24 could be fabricated directly on top of SU8 structures 23 as seen in FIG. 7. These structures could be, for example, 5 microns in height, and organized in geometric patterns as seen in FIGS. 15-23. The SU8 structure 23 could be an SU8-100 photoresist with a height of 75 microns, while flow channels 25 could be AZ 50XT photoresist with a height of 50 microns.

Each layer of photoresist could be patterned, fabricated, and aligned utilizing techniques common to those familiar with the art of photolithography. Such a mold could then be, for example, spin-coated with PDMS to create the middle layer 14 shown in FIG. 6. The total height could be, for example, 125 microns.

This fabrication protocol could be utilized to manufacture the various micro- and nano-substrates seen in FIGS. 15-23.

### Perfusion Layer

In some aspects, the single perfusion chamber described above can additionally include a perfusion layer where channels 27 in top layer 15, as seen in FIG. 8, are positioned directly above the specialized substrate region 12 to facilitate the flow of various gases, chemical compounds, and liquids in the local vicinity of the adhered cells. For example, the perfusion layer can feature a zig-zagging network of channels directly about substrate region 12. An inlet and/or outlet of the channels in the perfusion layer can be used to replenish the materials contained therein.

For example, gases such as CO2 can be introduced into the top perfusion layer via inlet 29 and, owing to the permeability of PDMS, can diffuse into the chamber below, enabling a suitable environment for cell growth. Similarly, chemical compounds such as growth factors, cytokines, amino acids can also be introduced into the perfusion layer and can diffuse through the PDMS membrane 28 separating the top and middle layers. This membrane can be, for example, tens of microns in thickness. This would be another strategy for refreshing the cell culture media without actually flushing the chamber.

Local temperatures can also be controlled via this top perfusion layer. By introducing fluids, for example water, of various temperatures, via heat transfer through the membrane 28 separating the top and middle layers 15 and 14 temperatures in the chamber below can also be controlled.

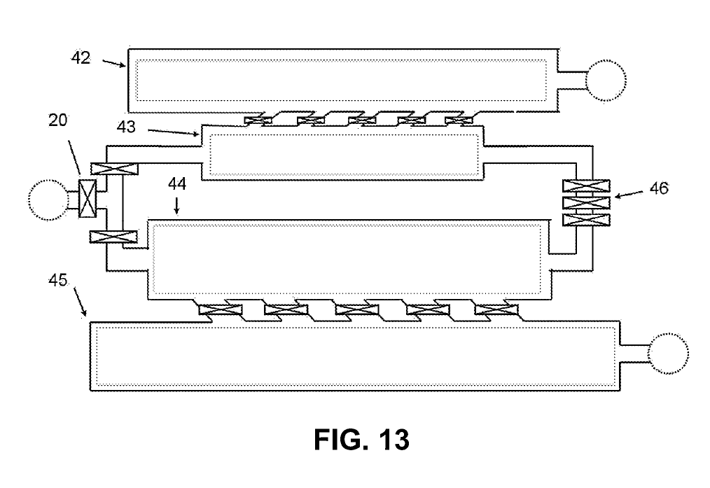

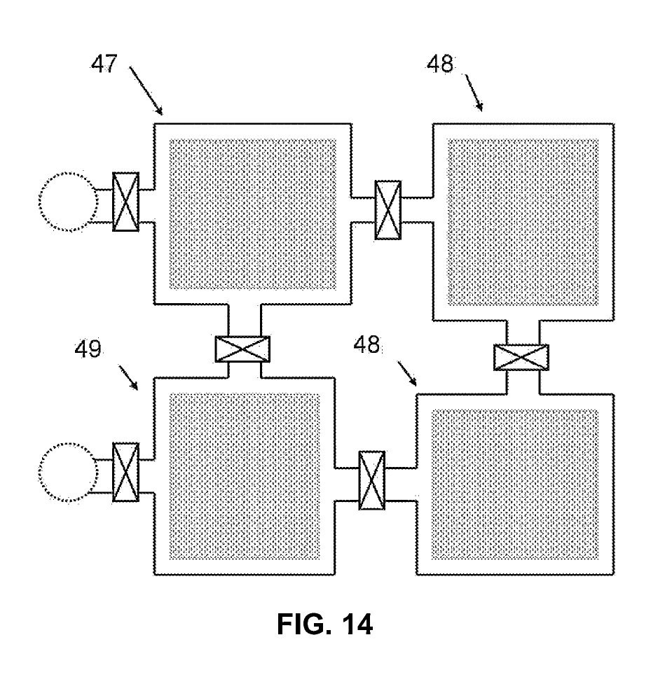

Using such local temperature control cells can be, for example, heat shocked and lysed. Boiling water introduced through the perfusion chamber could have such an effect. Once lysed, the cellular components can be introduced into the cell/protein/particle separation module shown in FIG. 13 and subsequently analyzed in the micro-ELISA module shown in FIG. 14.

Arrays featuring multiple perfusion channels can also be fabricated, as seen in FIG. 9.

Cell Adhesion, Release, and Manipulation. Perfusion Chamber Arrays.

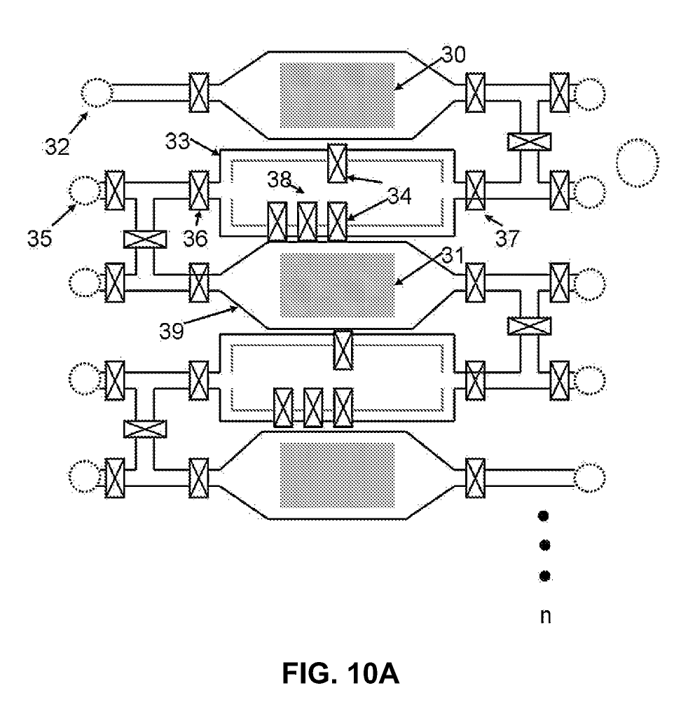

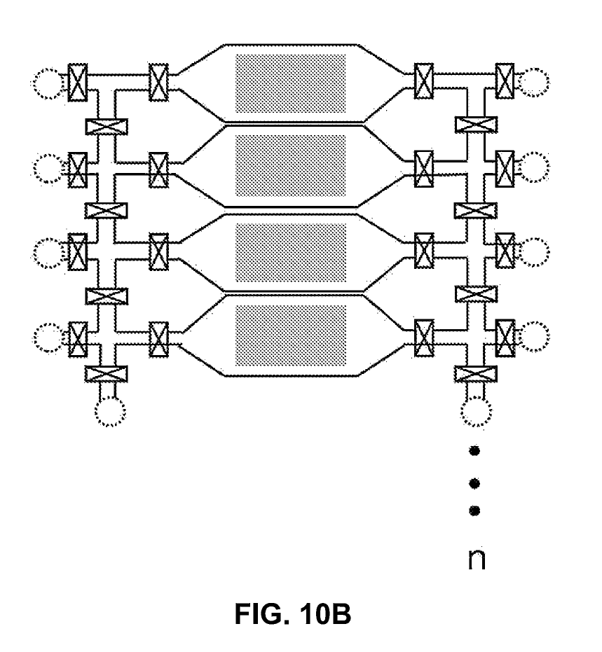

With reference now to FIG. 10, a number of modules are shown in series. Such an exemplary module can be used, for example, to study the interaction of the same cells on different micro- and nano-substrates. By way of example, as seen in FIG. 10A, cells can be introduced into the device such that they can adhere to substrate 30, examined via techniques such as optical-based microscopy, released and then adhered to substrate 31, examined, released again, and so on.

In such an embodiment, cells can be introduced into this module of the device via inlet 32, adhered to substrate 30 and inspected as described previously, at which point a trypsin solution can be introduced to release the cells into solution. This solution, now containing released cells, trypsin, and cell culture media, can then be introduced into mixer 33 and pressure loaded against valves 34. At this point, trypsin inhibitor can be introduced into the device via inlet 35 and pressure loaded against valves 34. Valves 36 and 37 can be sealed and the mixing valves 38 can be actuated, thus mixing together the cells, cell media, along with trypsin and trypsin inhibitor, thus de-activating the trypsin. Cells can also be washed with phosphate buffered solution (PBS) prior to trypsin treatment.

This new solution can then be introduced into chamber 39 with new micro-nano-substrate 31. The cells can then preferentially adhere to the new substrate 31, and once adhered, fresh cell culture media can be introduced into the chamber. At this point the cells can then be imaged and inspected again, yielding new data. This process can be repeated for any number of different substrates.

As seen in FIG. 10B, an array of perfusion chambers can also be utilized for the high-throughput analysis of cells on many different substrate types.

As noted above, all substrates can be coated and functionalized with protein coatings as described previously, for example, with glass “floors” being passivated with silane-PEG and cell adhesion surfaces being functionalized with one or more reagents for facilitating preferential adhesion of the cells to the surfaces such as fibronectin, collagen, laminin, or vitronectin.

### Metabolic Assays, Compound Discovery and Titration

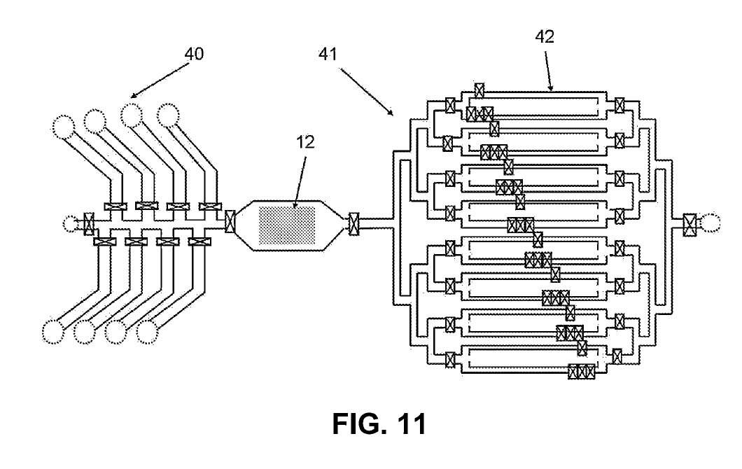

Cells adhered to these micro- and nano-substrates can also be studied and investigated utilizing metabolic assays. The exemplary embodiment depicted in FIG. 11 demonstrates a device where 8 different assay compounds can be introduced into the main reaction chamber via inlets 40 upon adhesion of the desired cells. As will be appreciated by a person skilled in the art, 8 channels are shown here, though more or less (e.g., n) channels are possible. Again, the cells can be introduced already dissociated from off-chip, or can be introduced from a dissociation module as described in FIGS. 1-5, or the separation module FIG. 13. As described above, the micro- and nano-substrate 12 can be functionalized with protein formulations while the glass surface can be passivated with a silane-PEG to prevent cell adhesion.

Using this same infrastructure shown in FIG. 11, for example, compound discovery can also be conducted by introducing various therapeutic molecules via inlets 40 which, upon interacting with cells adhered to substrate 12, can be monitored and biomarkers can be quantified for diagnostic use. In particular, arrays of substrates as shown in FIGS. 10A and 10B can be utilized such that therapeutic compounds can be tested with combinations of substrates 12, 30, 31, etc. By quantifying biomarkers in response to these various compounds, specific therapeutic compounds can be identified that can, for example, reduce the aggressiveness or oncogenic potential of a cancer, or reduce the invasion, motility, or metastatic potential of a cancer.

In addition to the channels for introducing metabolic assay compounds and compounds for therapeutic discovery, a titration module 41 for measuring the results of the metabolic assays can be included. With reference now to FIG. 11, an exemplary compound discovery system is depicted. Here, an initial metabolic assay compound A incubating with cells adhered to substrate 12 is introduced into the network of titration module 42 where, in each of the parallel mixers, a fraction of the sample is loaded. Each mixer can thus contain a different fraction of a first metabolic compound A—shown here to be ⅛ in mixer 1, ¼ in mixer 2, and so on. The remaining portion of each mixer can then be loaded with a metabolic assay compound B, and upon mixing of the two compounds, the resultant mixture can be imaged via microscopy techniques.

### Biomolecule (DNA) Transfection

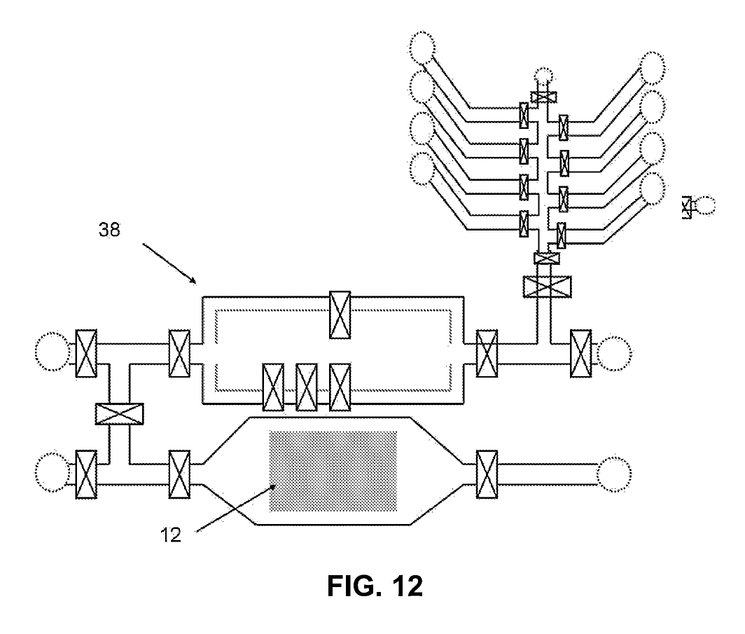

Cells adhered to these micro- and nano-substrates can also be studied and investigated utilizing metabolic assays. FIG. 12 depicts one exemplary device where 8 different assay compounds can be introduced into the main reaction chamber upon adhesion of the desired cells in the perfusion module. Again, the cells can be introduced already dissociated, or can be introduced from a dissociation module as described in FIGS. 1-5. As described above, the micro- and nano-cell adhesion substrate 12 can be functionalized with protein formulations while the glass surface can be passivated with a silane-PEG to prevent cell adhesion.

Input channels for introducing biomolecule compounds (i.e. nucleic acids) can flow into the mixing chamber 38 and can be incubated until cells are ready to be introduced. For example, an initial transfection compound A can be introduced to mixing chamber 38, and subsequently, additional transfection compounds (B, C etc.) can be introduced. After transfection reagents are mixed, transfection reagents from mixing chamber 38 can be flowed into the perfusion module having cells disposed on the cell adhesion substrate 12 and incubated in the perfusion module as necessary. After cells interact with transfection reagent and biomolecule, biomolecule can be incorporated into the intracellular space of the cells found on the adhesion substrate 12.

### Cell, Protein and Particle Sorter

With reference now to FIG. 13, a cell sorter module can also be provided. By way of example, upon completion of tissue disassociation, a suspension of cells and proteins can be introduced into the cell protein and particle sorter shown in FIG. 13. In this exemplary embodiment, the cell and protein suspension can circulate between mixing chambers 43 and 44. At an earlier time point, valves connecting chamber 43 to 42 can be open, allowing for particles ranging 0-10 microns in size to flow into chamber 42 and into another chamber for collection and/or analysis. At a later time point valves connecting chamber 44 and 45 can be opened to allow particles >10 microns to flow in chamber 45 and onto substrates designed to accommodate cells.

### Micro-ELISA

With reference now to FIG. 14, an ELISA module can also be provided. By way of example, proteins and small biomolecule fractions from the exemplary cell, protein and particle sorter discussed above can flow into one or more chamber(s) that have been functionalized with high affinity biomolecules such as, but not limited to, antibodies, nucleic acid aptamers, etc. In an exemplary embodiment, the antibodies used to functionalize chambers 47-48 can be directed at specific epitopes of proteins of interest. Upon flowing protein suspension into chamber 47, ligands specific for the antibody used to functionalize chamber 47 will bind the chamber surface via a direct interaction with the antibody. Now immobilized, other ligands within the protein suspension can be flowed into subsequent chambers, where other proteins can be immobilized by other antibodies already immobilized or used to functionalize the chamber. This process can be repeated with any number of chambers, antibodies and protein targets. After protein and antibody binding has taken place, chambers can be washed and treated with secondary antibodies, or fluorescent molecules to visualize the number or concentration of bound ligand.

### Substrate Descriptions:

Substrates with a plurality of topologies, geometries, protein coatings and extracellular environments can be designed and implemented to create specific areas of adhesion. These areas of adhesion will facilitate the acquisition of specific data points such as biomarkers or responses to therapeutic candidates. Each substrate will be located within a chamber designed for cell adhesion, culturing, growth and maintenance. All substrates can be coated and functionalized with protein coatings as described previously, and glass “floors” are also passivated with silane-PEG as described previously. By way of example, the cell adhesions surfaces could exhibit the following characteristics:

Micro gap bridging substrate, FIG. 15—To measure a cell's ability to bridge spatial gaps greater than 1 um, a substrate is designed to have planar surfaces of 10 microns in width separated by increasing gaps starting at 1 um, and increasing by 2 micron intervals up to 10 microns (50). Cells are introduced into the substrate, allowed to adhere and imaged at subsequent time points to assess the motility and morphology as they interact with the existing substrate. After cells are imaged via live microscopy, chemical agents to transfect, fix, permeabilized, and/or immunostaining the cells can be introduced to probe and identify subcellular structures within the cells.

Line bridging substrate, FIG. 16—To measure a cells ability to bridge spatial gaps greater than 1 um, a substrate is designed to have planar lines of 1 micron in width separated by increasing gaps starting at 1 um, and increasing by 2 um intervals up to 10 um (51). Cells are introduced into the substrate, allowed to adhere and imaged at subsequent time points to assess the motility and morphology as they interact with the existing substrate. After cells are imaged via live microscopy, chemical agents to transfect, fix, permeabilized, and/or immunostaining the cells can be introduced to probe and identify subcellular structures within the cells.

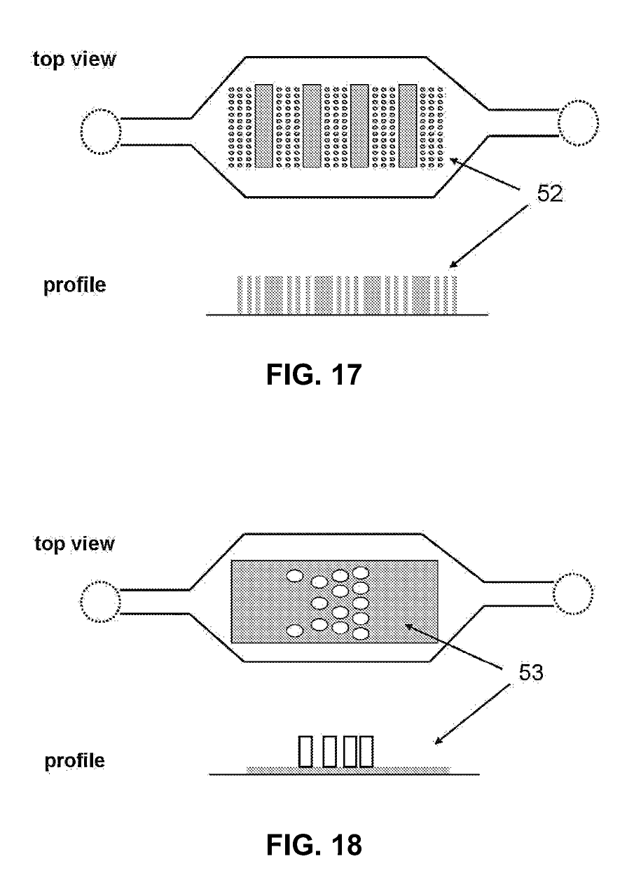

Protrusive force substrate, FIG. 17—To measure the forces cells are able generate at the leading edge, a substrate with planar surface of widths on the order of 10s of micrometers, adjacent to pillars with diameters on the orders of 100s of nanometers will be used (52). Cells are introduced into the substrate, allowed to adhere and imaged at subsequent time points to assess the motility and morphology as they interact with the existing substrate. After cells are imaged via live microscopy, chemical agents to transfect, fix, permeabilized, and/or immunostaining the cells can be introduced to probe and identify subcellular structures within the cells.

Macro-migratory potential substrate, FIG. 18—To measure a cells ability to migrate along a planar substrate with 3D obstacles, a planar substrate with columns of 20 um in diameter, spaced between 10 microns and 50 microns apart (53). Cells are introduced into the substrate, allowed to adhere and are imaged at subsequent time points to assess the motility and morphology as they interact with the existing substrate. After cells are imaged via live microscopy, chemical agents to transfect, fix, permeabilized, and/or immunostaining the cells can be introduced to probe and identify subcellular structures within the cells.

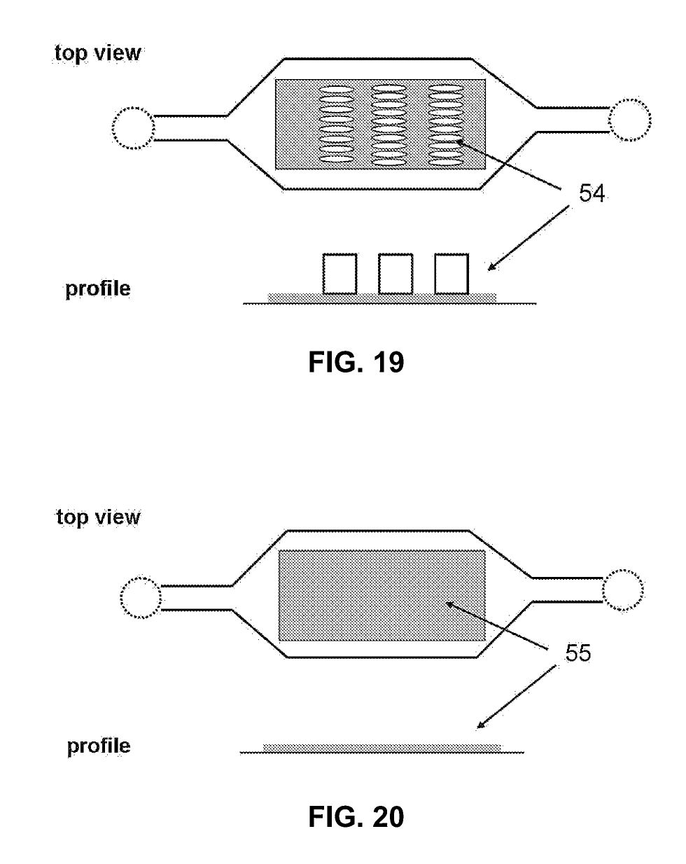

Micro-migratory potential substrate, FIG. 19—To measure a cells ability to migrate along a planar substrate with 3D obstacles, a planar substrate with columns of 10 um in diameter, spaced between 1 micron and 10 microns apart (54). Cells are introduced into the substrate, allowed to adhere and imaged at subsequent time points to assess the motility and morphology as they interact with the existing substrate. After cells are imaged via live microscopy, chemical agents to transfect, fix, permeabilized, and/or immunostaining the cells can be introduced to probe and identify subcellular structures within the cells.

Planar substrate, FIG. 20—To measure cell spreading and subcellular features in a planar environment, a flat substrate (55) the size of the chamber will allow cells to attach to the functionalized surface. Cells are introduced into the substrate, allowed to adhere and imaged at subsequent time points to assess the motility and morphology as they interact with the existing substrate. After cells are imaged via live microscopy, chemical agents to transfect, fix, permeabilized, and/or immunostaining the cells can be introduced to probe and identify subcellular structures within the cells.

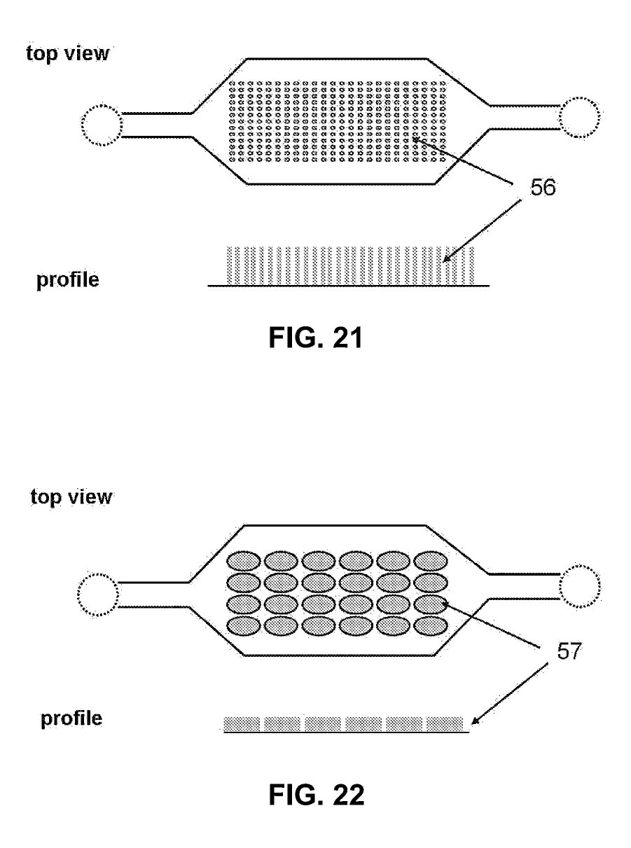

Micro Pillared substrate, FIG. 21—To measure cell spreading and subcellular features in a discontinuous environment, a substrate composed of the tops of pillars of 1 micron-5 microns heights and diameters of 0.5 micron-5 microns, spaced by 2× the diameter of the pillar (56) throughout the entire surface area of the chamber will allow cells to attach to the functionalized surface, deflecting posts and spanning multiple posts. Cells are introduced into the substrate, allowed to adhere and imaged at subsequent time points to assess the motility and morphology as they interact with the existing substrate. After cells are imaged via live microscopy, chemical agents to transfect, fix, permeabilized, and/or immunostaining the cells can be introduced to probe and identify subcellular structures within the cells.

Macro Pillared substrate, FIG. 22—To measure cell spreading and subcellular features in a discontinuous environment, a substrate composed of the tops of pillars of 10 microns-50 microns heights and diameters of 5 microns-50 microns, spaced by 2× the diameter of the pillar (57), throughout the entire surface area of the chamber will allow cells to attach to the functionalized surface, spanning multiple posts, bridging connections. Cells are introduced into the substrate, allowed to adhere and imaged at subsequent time points to assess the motility and morphology as they interact with the existing substrate. After cells are imaged via live microscopy, chemical agents to transfect, fix, permeabilized, and/or immunostaining the cells can be introduced to probe and identify subcellular structures within the cells.

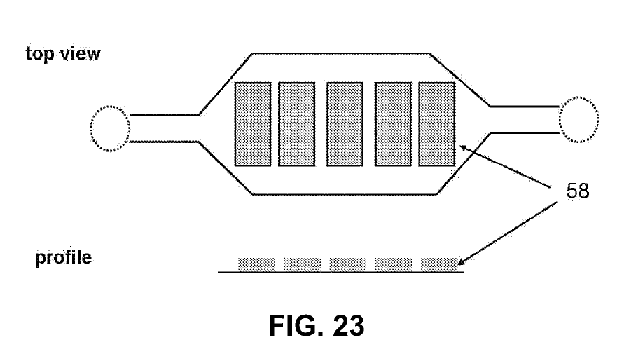

Macro gap bridging substrate. FIG. 23—To measure a cells ability to bridge spatial gaps greater than 10 microns, a substrate is designed to have planar surfaces of 10-100 microns in width separated by increasing gaps starting at 10 um, and increasing by 5 micron intervals up to 50 microns (58). Cells are introduced into the substrate, allowed to adhere and imaged at subsequent time points to assess the motility and morphology as they interact with the existing substrate. After cells are imaged via live microscopy, chemical agents to transfect, fix, permeabilized, and/or immunostaining the cells can be introduced to probe and identify subcellular structures within the cells.

### Device Integration

As noted above, though particular cell-processing functions are generally described with reference to individual cell-processing modules, it will be appreciated that the various exemplary modules and/or their functions can be integrated and/or combined to form a cell-processing system for performing multiple cell-processing functions. As will be appreciated by a person skilled in the art, all of the microfluidic device embodiments described above can be integrated in a modular fashion depending upon the desired applications of the device. By way of example, it will be appreciated that various exemplary independent modules described herein can be coupled to one another (e.g., in a lock-and-key manner) such that the microfluidic channels of each module can be coupled to one another. Alternatively, as will be discussed in detail below, various microfluidic cell-processing modules can be formed in a single monolithic structure (e.g., a microfluidic chip) to enable a specific clinical, diagnostic, and/or experimental workflow. Accordingly, the following description provides exemplary modules that can be incorporated into various systems in accord with the present invention.

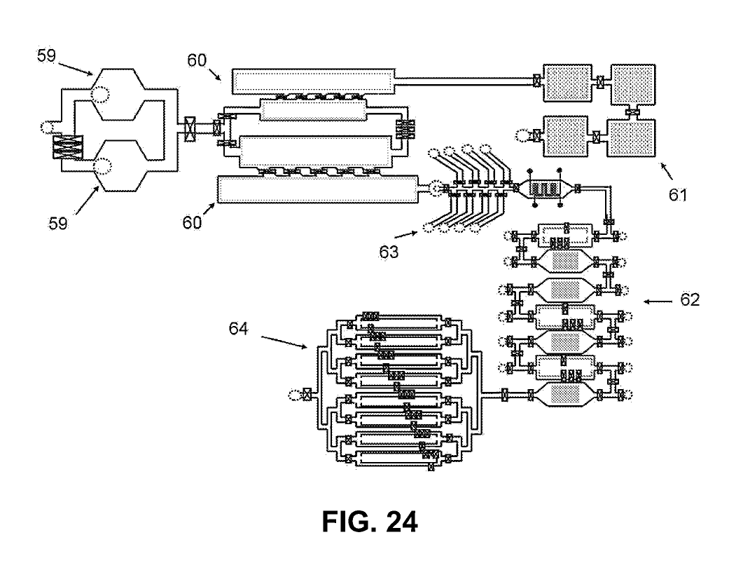

In an exemplary embodiment where all of the described modules are fully integrated as seen in FIG. 24, device operation can proceed by first introducing two tissue samples into dissociation chambers 59. Upon dissociation of the tissue into single cells, the cells can be segregated from proteins and other extra-cellular components in module 60, where separated components can next be assayed in the micro-ELISA module 61. Desired separated cells can then be loaded into the substrate array region 62 (any number of substrates are possible) where they can be adhered, inspected and manipulated amongst different substrates as described. Metabolic assays can be conducted by introducing the desired compounds from inlet channels 63, at which point the assay products can be inspected via the titration module 64. Similarly, various therapeutic compounds can also be introduced via inlet channels 63, whereupon the effects of these compounds can be monitored via microscopy techniques.

In such an embodiment, given the inputs of mammalian tissue, the device, in an automated, systematic fashion, can dissociate, segregate, sort, enrich, manipulate, and assay cells for biomarker quantification. These quantified biomarkers, which can be based on physical properties of the cells or biochemical/metabolic properties of the cells or associated extra-cellular components, can then be used as inputs into algorithms to output quantifiable metrics regarding the aggressiveness, or oncogenic potential, of a cancer, or the invasion, motility, or metastatic potential of a cancer. Examples of these algorithms can be found, for example in PCT/US2011/055444 filed Oct. 7, 2011, the contents of which are incorporated herein by reference.

As will be appreciated by a person skilled in the art in light of the teachings herein, the various exemplary modules can be utilized and integrated in various combinations depending upon the desired applications.

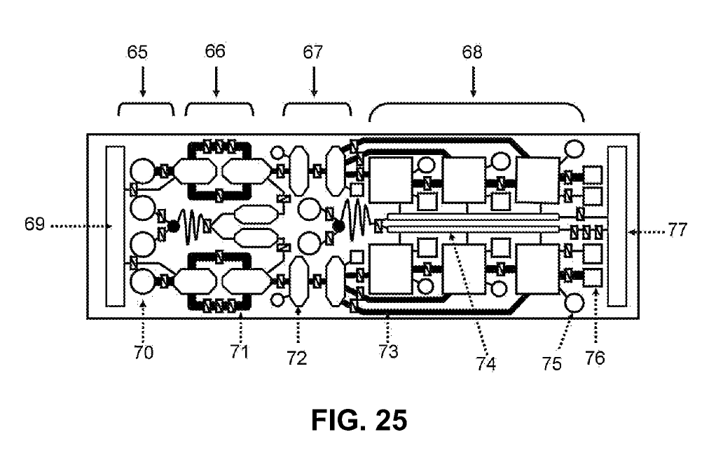

For example, in one exemplary embodiment, the described modules can be fully integrated using into a microfluidic system “chip” that can be used with existing microscopy platforms. For example, as schematically depicted in FIG. 25, device operation can proceed by first introducing two samples into zone 65, with inlets such as 70. Upon operation of various valves, samples can flow sequentially to zones 66, 67, and 68. Zone 66 represents an exemplary module capable of dissociating and decreasing size of particles. Zone 67 includes substrates that can enrich, separate, and/or supply samples with materials. Zone 68 includes channels that provide for distribution of samples to three independent regions. Reservoirs such as 69, 74, and 77 allow for storage of materials (e.g., cell culture media). Reservoirs 69, 74, and 77 can be accessible via diffusion or flow to various perfusion chambers (for example, but not limited to 71, 72, and 73). Inlets, 75 and outlets 76 can allow for continuous addition and removal of materials.

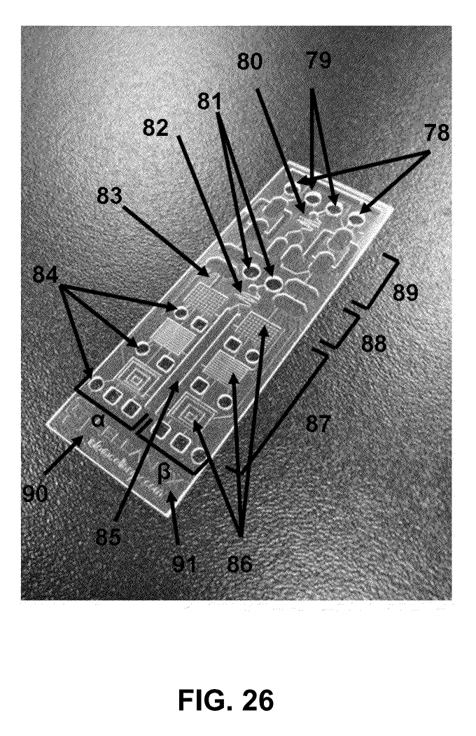

With reference now to FIG. 26, another exemplary workflow is described with reference to the depicted cell-processing system having various modules integrated on a single chip. As shown in FIG. 26, α and β samples can be run in parallel by inputting the necessary sample and reagents into the various sides 90, 91. By way of example, tissue fragments (e.g., minced fresh tissue, fresh-frozen tissue, frozen banked tissue) can be inserted into a cell mixture inlet 78, along with media and various proteases. After introducing the sample, the tissue fragments and/or cells are then transferred to the various downstream modules and subject to various cell processing procedures. As depicted, Zone 89 contains a dissociation module, Zone 88 contains a cell sorting module, and Zone 87 contains a plurality of cell adhesion modules that can be interrogated optically.

After introducing the tissue fragments and/or cells into the cell mixture inlet, the suspension can be circulated within the cell dissociation module 89 to equilibrate the mixture and dissociate large tissue fragments into a plurality of smaller tissue fragments and/or single cells. Using valves and pumps, the contents of the tissue dissociation module 89 can be transferred to the cell sorting module 88. By way of example, adhesion areas within the cell sorting module can be biofunctionalized to selectively capture specific cell types. Cells that do not adhere to these adhesions areas can flow, for example, into the various modules 86 of Zone 87 under the control of pumps and valves (not shown). In the depicted embodiment, each of the various cell adhesion substrates in the modules (86) can have different geometries and biofunctionalization, thus allowing for various analyses of cell characteristics and biomarkers as otherwise discussed herein and in PCT/US2011/055444 filed Oct. 7, 2011, the contents of which are incorporated herein by reference. For example, in various embodiments, Zone 87 can be imaged using standard fluorescent, brightfield and confocal microscopy.

The exemplary device additionally enables the addition of necessary reagents for performing the recited functions. By way of example, reagents to facilitate dissociation can be inserted into inlet 79 and mixed 80 prior to flowing into the dissociation module 89. Likewise, reagents necessary for culturing can be introduced into inlets 81, mixed in chamber 82, and stored in a reservoir 85 until needed to facilitate cell adhesion and culturing in Zone 87. As will be appreciated by one skilled in the art, other inlets and reservoirs can be included to allow for the delivery of various reagents. Ports in the shape of circles are inlets, and ports in shape of squares are outlets. For example, inlets (circles) and outlets (squares) 84 can be utilized to introduce and/or circulate new reagents into and out of the device.

One skilled in the art will appreciate further features and advantages of the presently disclosed methods, systems and devices based on the above-described embodiments. Accordingly, the presently disclosed methods, systems and devices are not to be limited by what has been particularly shown and described, except as indicated by the appended claims. All publications and references cited herein are expressly incorporated herein by reference in their entirety.

What is claimed is:

1. A system for cell processing, comprising:
at least one microfluidic cell dissociation module configured to dissociate said one or more tissue fragments received therein into one or more of single cells and smaller tissue fragments,
at least one microfluidic cell-processing module fluidly coupled to said at least one cell dissociation module for receiving at least a portion of said one or more single cells and smaller tissue fragments,
at least one reservoir in communication with at least one of said dissociation module and said cell-processing module, said reservoir being configured to store one or more reagents to be used within said dissociation module and said cell-processing module.

2. The system of claim 1, wherein said dissociation module comprises:
a first cell dissociation chamber having at least one inlet port for receiving one or more tissue fragments,
a channel fluidly coupled to said chamber to allow fluid to be circulated through the chamber, and
a pump coupled to any of said channel and said chamber to cause circulation of the fluid through said channel.

3. The system of claim 1, wherein said one or more cell-processing modules comprises:
an optically transparent layer having at least one portion transmissive to optical radiation,
a cell-processing layer defining a microfluidic channel for receiving a fluid having cells suspended therein, said cell-processing layer having one or more cell adhesion surfaces,
wherein said optically transmissive portion is positioned relative to said one or more cell adhesion surfaces to allow optical interrogation of cells adhered to said cell adhesion surfaces.

4. The system of claim 3, wherein said microfluidic channel of said cell-processing module is coupled one of directly or indirectly to a channel of said cell dissociation module.

5. The system of claim 3, wherein said cell-processing layer and said cell-dissociation module are formed in a monolithic substrate.

6. The system of claim 3, wherein the one or more cell-processing modules comprising at least a first and a second cell-processing modules connected in series, wherein each of said first and second cell-processing modules comprises at least one cell adhesion surface, and wherein a cell adhesion surface of the first cell-processing module differs from at least one of the cell adhesion surfaces of the second cell-processing module.

7. The system of claim 3, wherein the one or more cell-processing modules further comprise at least one inlet port for introducing one or more reagents into said microfluidic channel.

8. The system of claim 7, wherein said reagents facilitate at least one of metabolic assays and compound discovery.

9. The system of claim 7, further comprising a titration module.

10. The system of claim 3, wherein the one or more cell-processing modules comprise at least a first and a second cell-processing module connected in parallel.

11. The system of claim 3, further comprising a sorter module fluidly coupled to the cell dissociation module and cell-processing module and disposed therebetween.

12. The system of claim 11, wherein the cell sorter module is configured to discriminate particles based on size.

13. The system of claim 12, further comprising an ELISA module fluidly coupled to said cell sorter module such that particles having a diameter less than about 10 microns are diverted to said ELISA module.

14. The system of claim 13, wherein said ELISA module comprises one or more surfaces functionalized with high affinity biomolecules.

15. (canceled)

16. The system of claim 1, further comprising an imager configured to interrogate cells within said cell-processing module.

17. The system of claim 16, wherein the imager is configured to image a cell adhesion surface of said cell-processing module with one of fluorescence, confocal, differential interference contrast, and total internal reflection fluorescence microscopy.

18. A microfluidic device for processing tissue, comprising:
a first cell dissociation chamber having at least one inlet port for receiving one or more tissue fragments,
a channel fluidly coupled to said chamber to allow fluid to be circulated through the chamber, and
a pump coupled to any of said channel and said chamber to cause circulation of the fluid through said channel.

19. The device of claim 18, wherein said channel has a cross-sectional area in a range of about 10 microns to about 1000 microns.

20. The device of claim 18, further comprising a plurality of microstructures disposed is said channel so as to facilitate dissociation of said one or more tissue fragments.

21. The device of claim 20, wherein the microstructures are pyramidal.

22. The device of claim 18, wherein said channel comprises at least one inlet port for introducing one or more reagents into said channel.

23. The device of claim 22, wherein said one or more reagents are configured to facilitate dissociation of said one or more tissue fragments.

24. The device of claim 23, wherein said one or more reagents comprise a protease selected from the group consisting of trypsin, DNase, papain, collagenase type I, II, III, IV, hyoluronidase, elastase, protease type XIV, pronase, dispase I, dispase II, and neutral protease.

25. The device of claim 18, further comprising a second dissociation chamber fluidly coupled to said first dissociation chamber and said channel so as to provide a closed loop fluid circulating path.

26. (canceled)

27. (canceled)

28. A microfluidic device, comprising
an optically transparent layer having at least one portion transmissive to optical radiation,
a cell-processing layer defining a microfluidic channel for receiving a fluid having cells suspended therein, said cell-processing layer having one or more cell adhesion surfaces,
wherein said optically transmissive portion is positioned relative to said one or more cell adhesion surfaces to allow optical interrogation of cells adhered to said cell adhesion surfaces.

29. The microfluidic device of claim 28, further comprising a perfusion layer coupled to said cell processing layer, said perfusion layer comprising one or more channels disposed therein and positioned relative to said one or more cell adhesion surfaces so as to allow diffusion of any of a gas and a nutrient to cells adhered to said one or more cell adhesion surfaces.

30. (canceled)

31. The microfluidic device of claim 28, wherein said one or more cell adhesion surfaces are functionalized with one or more reagents suitable for facilitating preferential adhesion of cells to said surfaces.

32. The microfluidic device of claim 31, wherein said one or more reagents comprise at least one of fibronectin, collagen, laminin, and vitronectin.

33. (canceled)

34. The microfluidic device of claim 28, wherein the cell-processing layer comprises thermoplastics, thermosets, and elastomers such as epoxy, phenolic, PDMS, glass, silicones, nylon, polyethylene, polysterene.

35. The microfluidic device of claim 28, wherein at least one surface of said optically transparent portion is functionalized with one or more reagents suitable to prevent adhesion of cells to said surface.

36. (canceled)

37. The microfluidic device of claim 28, wherein said one or more cell adhesion surfaces are substantially planar.

38. The microfluidic device of claim 28, wherein said one or more cell adhesion surfaces comprises one or more microstructures.

39-42. (canceled)

43. A method of processing tissue, comprising:
introducing one or more tissue fragments into a microfluidic cell dissociation module such that said one or more tissue fragments dissociate into any of single cells and smaller tissue fragments,
transferring at least a portion of said single cells and/or smaller tissue fragments to a microfluidic cell-processing module fluidly coupled to said at least one cell dissociation module,
processing said cells and/or smaller tissue fragments such that said cells adhere to one or more cell adhesion surfaces of said microfluidic cell-processing module, and
imaging at least a portion of said cells adhered to one of said cell adhesion surfaces.
